(19) United States
(12) Patent Application Publication
(10) Pub. No.: US 2019/0015306 A1
(43) Pub. Date: Jan. 17, 2019

(54) PACK COMPOSITION CONTAINING ILLITE EXTRACT AS ACTIVE INGREDIENT AND PACK CONTAINING SAME

(71) Applicant: Soung Lyul PARK, Daejeon (KR)

(72) Inventors: Soung Lyul PARK, Daejeon (KR); Jung Woo PARK, Daejeon (KR)

(73) Assignee: Soung Lyul PARK, Daejeon (KR)

(21) Appl. No.: 16/066,894

(22) PCT Filed: Aug. 23, 2016
(86) PCT No.: PCT/KR2016/009272

§ 371 Date: Jul. 17, 2018

(30) Foreign Application Priority Data
Dec. 31, 2015 (KR) 10-2015-0191323

(51) Int. Cl. A61K 8/26; A45D 44/00; A61K 8/9789; A61Q 19/02; A61Q 19/08; A61K 8/9794

(52) U.S. Cl. CPC A61K 8/26; A45D 44/002; A61K 8/9794; A61Q 19/02; A61Q 19/08; A61K 8/9789

(57) ABSTRACT

The present invention relates to a pack composition containing an illite extract as an active ingredient. The pack composition consists of: a first agent comprising, with respect to 85.76 parts by weight of an illite extract, 2 parts by weight of carbomer, 0.04 parts by weight of adenosine, 1.2 parts by weight of caffeine, 1.2 parts by weight of β-glucan, 0.6 parts by weight of an oriental raisin fruit extract, 0.6 parts by weight of a green tea extract, 0.6 parts by weight of a whitening composition, 0.5 parts by weight of a black bean extract, 0.5 parts by weight of an oregano extract, 0.2 parts by weight of a Scutellaria baicalensis extract, 0.2 parts by weight of a sophora root extract, 3.0 parts by weight of acetyl hexapeptide-8, 2.0 parts by weight of copper tripeptide-1, 1.0 part by weight of an oat kernel extract, 0.3 parts by weight of dermosoft® Octiol and 0.3 parts by weight of phenoxyethanol; and a second agent comprising, with respect to 53.8 parts by weight of the illite extract, 1 part by weight of a Portulaca oleracea extract, 1 part by weight of a green tea extract, 2 parts by weight of a white flower complex, 2 parts by weight of sodium hydroxide (NaOH), 0.2 parts by weight of polyepsilon-lysine, and 40 parts by weight of sodium silicate, wherein the first agent and the second agent are mixed at a ratio of 1:1.

### TECHNICAL FIELD

The present invention relates to a cosmetic pack composition containing and more particularly, to a cosmetic pack composition containing, as a main ingredient, an illite extract to selectively control pH.

More specifically, the present invention relates to a pack composition containing an illite extract as an active ingredient which includes a first formulation including, based on 85.76 parts by weight of the illite extract, 2 parts by weight of carbomer, 0.04 parts by weight of adenosine, 1.2 parts by weight of caffeine, 1.2 parts by weight of β-glucan, 0.6 parts by weight of a Hovenia dulcis fruit extract, 0.6 parts by weight of a green tea extract, 0.6 parts by weight of a whitening composition, 0.5 parts by weight of a black soybean extract, 0.5 parts by weight of an Oreganum vulgare extract, 0.2 parts by weight of a Scutellaria baicalensis extract, 0.2 parts by weight of a Sophora angustifolia extract, 3.0 parts by weight of acetyl hexapeptide-8, 2.0 parts by weight of copper tripeptide-1, 1.0 part by weight of an Avena sativa (Oat) kernel extract, 0.3 parts by weight of octanediol (dermosoft® Octiol) and 0.3 parts by weight of phenoxyethanol, and

a second formulation, including, based on 53.8 parts by weight of the illite extract, 1 part by weight of a Portulaca oleracea extract, 1 part by weight of a green tea extract, 2 parts by weight of a white flower complex, 2 parts by weight of sodium hydroxide (NaOH), 0.2 parts by weight of poly-epsilon-lysine and 40 parts by weight of sodium silicate,

wherein the pack composition is provided by mixing the first formulation with the second formulation in a ratio of 1:1.

According to the present invention, skin health can be promoted owing to skin antioxidant activity or the like, texture can be enhanced and in particular, excellent skin whitening can be obtained.

### BACKGROUND ART

In general, cosmetics function to keep the skin clean and beautiful, to maintain healthy skin, and to delay or prevent skin aging expressed physiologically over time.

As the skin becomes older, the lipid composition and content of the lipid layer serving as a skin barrier change and its functions are rapidly deteriorated, which results in a decrease in moisture content of the skin, skin dryness and various skin lesions.

In addition, the increased amount of ultraviolet ray due to environmental pollution, air pollution and fatigue and stress of modern people and the like have brought about production of active oxygen, free radicals and peroxides each having lower reactivity, which affect the skin, for example, facilitating skin aging while causing oxidation or denaturation of biological ingredients (proteins, nucleic acids and cell membrane lipids).

Particularly, collagen, elastin and the like that constitute connective tissues of the skin are oxidized and cease to function, so that the skin loses its elasticity and is converted into senile skin along with formation of excessive wrinkles.

Such skin lesions or aging are due to non-homogenization of cells, loss of elastin, destruction of collagen, and reduction and delay of collagen synthesis, and eventually skin aging occurs even in the epidermis, but mostly in the dermis.

Accordingly, among various conventional methods of reducing or delaying skin aging, the best method is to apply packs or the like to the skin for a prolonged period of time so that effective ingredients that can reduce and/or delay skin aging can be introduced into the epidermal layer of the skin.

There are technologies to accomplish such a method. For example, Korean Patent No. 10-0904552 (hereinafter, referred to as “Prior art 1”) discloses a cosmetic composition for skin lifting.

Prior art 1 discloses a cosmetic composition for skin lifting that includes: a first formulation including purified water, a thickening agent, an antimicrobial agent and a skin-enhancing ingredient; and a second formulation including sodium silicate, sodium hydroxide and an antimicrobial agent, and optionally including organogermanium, wherein the cosmetic composition exhibits low skin irritation, convenient use and excellent lifting effects when using a combination of the first formulation and the second formulation.

Meanwhile, as described above, with the recent trend of lookism (appearance-oriented views) in addition to the desire to maintain the health of the skin through skin lifting and anti-aging, there is an increasing desire for consumption to maintain white skin regardless of gender and age.

Maintenance of white skin can be accomplished by using a composition with lightening effect and Prior art 1 does not disclose skin whitening.

However, regarding skin whitening, Korean Patent No. 10-1081059 (hereinafter, referred to as “Prior art 2”) with antioxidant and whitening activities discloses a flower mixture extract, a method for extracting the same and a cosmetic composition containing the flower mixture extract.

Prior art 2 can improve skin antioxidant activity and whitening activity by using, as a flower mixture extract with antioxidant and whitening activities, an extract obtained by extracting, in water, an organic solvent or a mixture thereof, a flower mixture of two or more of Adonis amurensis, Akebia quinata, Albizia julibrissin, Althaea rosea, Angelica acutiloba, Angelica anomala, Aquilegia buergeriana, Artemisa princeps, Aster ciliosus, Aster scaber, Brassica campestris, Broussonetia kazinok, Callistephus chinensis, Camellia japonica, Camellia sinensis, Campsis grandiflora, Caragana sinica, Carthamus tinctorius, Cassia obtusifolia, Celosia cristata, Cercis chinensis, Chaenomeles sinensis, Chrysanthemum indicum, Chrysanthemum zawadskii, Clematis turniflora, Commelina communis, Cornus kousa, Cornus officinalis, Corydalis incisa, Corydalis turtschaninovii, Cucurbita moschate, Dendrobium nobile, Dianthus chinensis, Elsholtzia splendens, Eupatorium lindleyanum, Forsythia koreana, Fritillaria ussuriensis, Gardenia jasminoides, Ginkgo biloba, Helianthus annuus, Helianthus tuberosus, Hemerocallis fulva, Hibiscus mutabilis, Hibisicus syriacus, Hosta plantaginae, Hydrangea macrophylla, Impatiens balsamina, Iris pallasii, Iris pseudacorus, Kerria japonica, Lagerstroemia indica, Leonurus sibiricus, Lespedeza bicolor, Lilium lancifolium, Lindera obtusiloba, Liriope muscari, Lonicera japonica, Lycoris radiata, Magnolia kobus, Magnolia liliflora, Malus sieboldii, Narcissus tazetta, Nelumbo nucifera, Oenothera biennis, Paeonia lactiflora, Paeonia suffruticosa, Papaver rhoeas, Patrinia scabiosifolia, Paulownia coreana, Persicaria thunbergii, Petasites japonicus, Ipomoea purpurrea L., Phytolacca esculenta, Platycodon grandiflorus, Polygonatum odoratum, Poncirus trifoliata, Prunus armeniaca, Prunus mume, Prunus persica, Prunus serrulata, Prunus tomentosa, Pueraria lobata, Rhododendron mucronulatum, Robinia pseudacacia, Rosa multiflora, Rosa rugosa, Sasa borealis, Sophora japonica, Spiraea prunifolia, Styrax japonica, Syringa vulgaris, Taraxacum platycarpum, Tulipa edulis, Viola mandschurica, Weigela subsessilis, Wisteria sinensis, Zanthoxylum schinifolium, Zea mays, Zingiber mioga, and Zinnia elegans.

However, neither Prior art 2 nor Prior art 1 discloses an illite composition.

Since the illite composition imparts excellent effects such as moisturizing, elasticity, whitening and cleaning effects to the skin, as described below, the present applicant completed the present invention by introducing an illite composition and using the illite composition as an illite extract by a method capable of controlling pH of the illite composition.

Also, in particular, Prior art 2 discloses a white flower composition and fails to prove effectiveness of ingredients of the white flower composition, although some effective ingredients of the composition may be changed or lost according to the processing method.

On the other hand, the present applicant tested the possibility that the ingredients of the composition can be effective when used in conjunction with the aforementioned illite extract and provided a composition effective for skin whitening by determining the content of the white flower composition, with respect to the illite extract, based on test results.

### DISCLOSURE

### Technical Problem

Therefore, the present invention has been made in view of the above problems, and it is an object of the present invention to provide a pack composition containing an illite extract as an active ingredient, which can provide a composition suitable for various types of skin depending on controlled pH, when used as a pack, by providing a pack composition that essentially includes an illite extract to selectively control pH, and can improve skin lightening through a white flower composition and a whitening composition present in the composition.

### Technical Solution

In accordance with the present invention, the above and other objects can be accomplished by the provision of a pack composition containing an illite extract as an active ingredient, including, a first formulation including, based on 85.76 parts by weight of the illite extract, 2 parts by weight of carbomer, 0.04 parts by weight of adenosine, 1.2 parts by weight of caffeine, 1.2 parts by weight of β-glucan, 0.6 parts by weight of a Hovenia dulcis fruit extract, 0.6 parts by weight of a green tea extract, 0.6 parts by weight of a whitening composition, 0.5 parts by weight of a black soybean extract, 0.5 parts by weight of an Oreganum vulgare extract, 0.2 parts by weight of a Scutellaria baicalensis extract, 0.2 parts by weight of a Sophora angustifolia extract, 3.0 parts by weight of acetyl hexapeptide-8, 2.0 parts by weight of copper tripeptide-1, 1.0 part by weight of an Avena sativa (Oat) kernel extract, 0.3 parts by weight of octanediol (dermosoft® Octiol) and 0.3 parts by weight of phenoxyethanol; and a second formulation, including, based on 53.8 parts by weight of the illite extract, 1 part by weight of a Portulaca oleracea extract, 1 part by weight of a green tea extract, 2 parts by weight of a white flower complex, 2 parts by weight of sodium hydroxide (NaOH), 0.2 parts by weight of poly-epsilon-lysine and 40 parts by weight of sodium silicate, wherein the first formulation and the second formulation are mixed in a ratio of 1:1.

### Advantageous Effects

The pack composition containing an illite extract as an active ingredient according to the present invention and the pack produced from the composition can promote skin health owing to skin antioxidant activity or the like, enhance texture and, in particular, provide excellent skin whitening.

### DESCRIPTION OF DRAWINGS

The above and other objects, features and other advantages of the present invention will be more clearly understood from the following detailed description taken in conjunction with the accompanying drawings, in which:

FIG. 1 shows results of measurement tests associated with tyrosinase inhibition effect of a white flower complex in a pack composition containing an illite extract as an active ingredient according to the present invention; and

FIG. 2 shows test results demonstrating that, regarding DPPH free radical scavenging activity of the white flower complex in the pack composition containing an illite extract as an active ingredient according to the present invention, the feature that the effective ingredients of the white flower complex have an effectiveness probability of 50% relates to the content of the white flower complex.

### BEST MODE

All terms or words used in the specification and claims should not be limited to common or dictionary meanings and should be interpreted as having a meaning and concept that are consistent with the technical idea of the present invention, based on the principle that the present inventors can appropriately define the concept of the terms in order to explain the present invention in the best way.

Accordingly, Test example and Reference Example disclosed herein are provided only for most preferred embodiments of the present invention and do not represent all technical concepts of the present invention. Accordingly, it should be understood that there are a variety of equivalents and modifications that can replace them at the time of filing.

The present invention relates to a cosmetic pack composition that essentially includes an illite extract to selectively control pH.

More specifically, the present invention relates to a pack composition containing an illite extract as an active ingredient which includes: a first formulation including, based on 85.76 parts by weight of the illite extract, 2 parts by weight of carbomer, 0.04 parts by weight of adenosine, 1.2 parts by weight of caffeine, 1.2 parts by weight of β-glucan, 0.6 parts by weight of a Hovenia dulcis fruit extract, 0.6 parts by weight of a green tea extract, 0.6 parts by weight of a whitening composition, 0.5 parts by weight of a black soybean extract, 0.5 parts by weight of an Oreganum vulgare extract, 0.2 parts by weight of a Scutellaria baicalensis extract, 0.2 parts by weight of a Sophora angustifolia extract, 3.0 parts by weight of acetyl hexapeptide-8, 2.0 parts by weight of copper tripeptide-1, 1.0 part by weight of an Avena sativa (Oat) kernel extract, 0.3 parts by weight of octanediol (dermosoft® Octiol) and 0.3 parts by weight of phenoxyethanol; and a second formulation, including, based on 53.8 parts by weight of the illite extract, 1 part by weight of a Portulaca oleracea extract, 1 part by weight of a green tea extract, 2 parts by weight of a white flower complex, 2 parts by weight of sodium hydroxide (NaOH), 0.2 parts by weight of poly-epsilon-lysine and 40 parts by weight of sodium silicate, wherein the pack composition is provided by mixing the first formulation with the second formulation in a ratio of 1:1.

The present invention will be described in detail below.

### EXAMPLE 1

### Pack Composition Containing Illite Extract

The pack composition according to an embodiment of the present invention broadly includes a first formulation and a second formulation, wherein a mixture of the first formulation and the second formulation in a ratio of 1:1 is used for the pack composition.

The first formulation may include, as a skin enhancer (with lifting and lightening effects), based on 85.76 parts by weight of an illite extract, 2 parts by weight of carbomer, 0.04 parts by weight of adenosine, 1.2 parts by weight of caffeine, 1.2 parts by weight of β-glucan, 0.6 parts by weight of a Hovenia dulcis fruit extract, 0.6 parts by weight of a green tea extract, 0.6 parts by weight of a whitening composition, 0.5 parts by weight of a black soybean extract, 0.5 parts by weight of an Oreganum vulgare extract, 0.2 parts by weight of a Scutellaria baicalensis extract, 0.2 parts by weight of a Sophora angustifolia extract, 3.0 parts by weight of acetyl hexapeptide-8, 2.0 parts by weight of copper tripeptide-1, 1.0 part by weight of an Avena sativa (Oat) kernel extract, 0.3 parts by weight of octanediol (dermosoft® Octiol) and 0.3 parts by weight of phenoxyethanol.

The illite extract used herein was one buried in Youngdong-gun, Chungbuk and was extracted based on Reference Example 1 described later using rare mineral called “illite”.

This applies the same to the illite extract used for the second formulation.

Illite is hydrous mica produced by dehydration of mica minerals that belong to phyllosilicate (layered silicate) during weathering, or leaching of K⁺, Mg²⁺ and the like, and the representative chemical formula thereof is {K_{0.75} [Al_{1.75}(MgFe²⁺)_{0.25}] (Si_{3.50}Al_{0.50})O₁₀(OH)₂}.

Illite adsorbs suspended solids in water, and is anionic and thus electrically neutralized with fine cationic suspended particles to induce coagulative precipitation and thereby make water clean.

In addition, illite is capable of adsorbing, deodorizing and degrading heavy metals and harmful gas, is capable of generating anions and far infrared rays at room temperature, has antibacterial and antiviral activities, strengthens immunity in animals, and is effective in treating certain diseases and facilitating growth.

In particular, illite is a mineral that emits soft far-infrared light, is safe for skin, and exhibits not only physiological effects on cells, but also excellent cosmetic effects such as moisturizing, elasticity, whitening and cleaning effects.

Illite is also effective in relieving acne, sebum and atopy, is richer in minerals than yellow clay, has excellent heat and exfoliation effects, and is effective in removing lead, heavy metals and toxins.

In addition, illite has excellent ability to absorb and remove various waste such as environmental hormones, generates a great amount of anions to provide a forestation effect, and clarifies the skin through antibacterial action to alleviate acne and atopic dermatitis.

Illite contains ingredients such as silicon dioxide (SiO₂), alumina (Al₂O₃), iron oxide (Fe₂O₃) calcium oxide (CaO), sodium oxide (Na₂O) and magnesium oxide (MgO), and silicon dioxide is present therein in the greatest amount.

Silicon oxide acts to remove waste and excess sebum in pores, alumina acts to promote blood circulation, iron oxide acts on collagen binding, calcium oxide acts to detoxify and inhibit stress, sodium oxide acts to regulate water and osmotic pressure, and magnesium oxide acts to promote waste excretion.

Carbomer is a thickening agent to increase the viscosity of the first formulation, which is a skin enhancer and is preferably carbopol 934, but the present invention is not limited thereto.

In addition, adenosine, caffeine, β-glucan, Hovenia dulcis fruit extract, green tea extract, the whitening composition, the black soybean extract, the Oreganum vulgare extract, the Scutellaria baicalensis extract, the Sophora angustifolia extract, acetyl hexapeptide-8, copper tripeptide-1, the Avena sativa (Oat) kernel extract, octanediol (dermosoft® Octiol) and phenoxyethanol each have an effect of improving skin lifting.

Among them, the whitening composition may be any one of a Centella asiatica extract, a Diospyros kaki leaf extract, a Theobroma cacao (cocoa) extract, a chamomile extract and a wine extract, and methods for obtaining each extract are ordinary extraction methods and the detailed explanation thereof is thus omitted.

The Centella asiatica extract originates from the Indian tropics and is a plant ideally growing at 600 meters above the sea level in moist and shady environments, which is also called “Gotu kola”, “Indian pennywort” or “Tiger grass”.

Such a Centella asiatica extract contains asiaticoside, madecasosside, asiatic acid and madecassic acid, each which are called triterpenoids (saponins), and are known to function to control and promote collagen synthesis of the human body, to strengthen blood vessels and thereby to promote blood circulation, and to help improve memory.

The Diospyros kaki leaves are derived from the leaf part of Diospyros kaki, and Diospyros kaki is a rare chemical-free fruit tree that grows well without using a chemical. The leaves are known as a repository of vitamin C.

These leaves contain ascorbic acid and exhibit anti-oxidative and whitening effects owing to vitamin C.

In addition, the Diospyros kaki leaves induce synthesis of collagen that maintains skin elasticity, prevents aging and eliminates the melanin pigment which causes spots and freckles.

In addition, tannins contained in the Diospyros kaki make the skin elastic and smooth, because they act on hemostatic convergence. In addition, Diospyros kaki leaves exhibit anti-inflammatory and antibacterial effects, heal wounds and remove germs to contribute to clean and healthy skin owing to flavonoid glycoside and phenolic coumarin compounds.

The Theobroma cacao (cocoa) extract is obtained by harvesting seeds from ripe berries, fermenting the same in a wooden bottle for several days to obtain the seeds that turn into reddish brown and emit a specific cacao scent, washing the seeds with water, drying, and roasting the resulting Cacao beans and powderizing the same to produce a cacao paste.

Such cacao contains ingredients such as theobromine, sugar, epicatechin, catechin, tannin and cacao polyphenols, these ingredients inhibit active oxygen to prevent skin aging and help skin whitening.

In addition, cacao polyphenols have immunomodulatory function and thus help to protect the skin from the external environment such as ultraviolet light and to regenerate damaged skin tissues owing to allergy suppression and antioxidant activity.

Chamomiila recutitia (matricaria) is a member of the family Asteraceae and is free from damage by blight and harmful insects, because insects do not like sweetness and apple flavors emitted by these chamomile flowers. These chamomile is beneficial not only to humans, but also plants. More specifically, chamomile has a nickname of “plant physician”, because of the saying that plants infected with pests can be revitalized when planting chamomile next thereto.

Chamomiila recutitia contains ingredients such as caffeic acid, catechin tannin, chamomillol, chiline, Kaempferol, luteolin, niacin, palmitic acid, pectic acid, rutin and thiamin, and these ingredients are known to have anti-inflammatory activity to heal wounds and promote tissue regeneration, in particular, to be remarkably effective in burns.

In addition, Chamomiila recutitia improves skin elasticity, is effective for treating dry and itchy skin, acts as an antimicrobial agent, protects the skin from bacterial infection or irritation by substances in the air, and is less toxic than other herbs.

The wine extract is prepared by aging whole grapes for a prolonged time and red grapes are known to have excellent antioxidant activity, because the skin and seeds of red grapes contain great amounts of polyphenol ingredients.

Such red wine contains thiamin, riboflavin, niacin, pantothenic acid, vitamin B6, B12, Ca, Fe, Mg, PK, Na, Zn, Cu, Mn, Se, anthocyanins, proanthocyanidins, polyphenols and the like, polyphenol ingredients inhibit formation of melanin, prevent formation of stains, freckles and the like. Red wine contains a great amount of anthocyanin, which is stronger antioxidant activity than tocopherol, and thus exhibits antimicrobial, skin soothing and skin elasticity effects.

Some ingredients present in red wine may be changed because red wine can be prepared by a variety of methods. It should be understood that wine containing the ingredients prepared by an ordinary method is preferably used.

In addition, the black soybean extract (Glycine soja seed extract) contains more than twice as much protein as ordinary beans, contains 18 types of amino acids and 8 types of essential amino acids, and is rich in lecithin.

Such lecithin is a substance that regulates moisture in cells, imparts shine and gloss to the skin and aids absorption of fat-soluble vitamins including vitamin E to prevent skin aging and pigmentation. As such, black beans facilitate blood circulation even up to peripheral parts of the human body to supply nutrition and oxygen to the skin and thereby make the skin clear and healthy.

In addition, beta-glucan is a polysaccharide contained in the cell walls of mushrooms, yeast and grains.

Since beta-glucan extracted from shiitake mushrooms was already introduced as an anti-cancer drug “Lentinan” in Japan in the 1980s, a variety of beta-glucan extract products extracted from Schizophyllum commune, Sparassis crispa, Trametes versicolor, Grifola frondos and the like have been released.

Use of such mushroom-derived beta-glucan is expanding to a new moisturizing substance which is eco-friendly and utilizes conversion to low-molecular weight materials (fermentation, nanomaterials, etc.). The mushroom-derived beta-glucan attracts a great deal of attention as a third generation cosmetic moisturizing ingredient since it is found to have a 20% better moisturizing function of controlling skin oil and moisture than hyaluronic acid.

In addition, acetyl hexapeptide-8 is a non-toxic and harmless substance that attracts great attention as a substitute for cosmetics that eliminate concerns about side effects (such as allergic reactions, facial muscle paralysis, lid ptosis, etc.). When acetyl hexapeptide-8 is applied as a raw material for cosmetics, it is harmless to the environment and human health.

Such acetylhexapeptide-8 is a low-molecular weight peptide having similar functions to conventional botulinum toxin, which increases skin elasticity, and improves beauty and anti-aging function by regulating the signal transduction process of neuron cells connected to wrinkle muscles.

In addition, acetylhexapeptide-8 is a low-molecular weight peptide that easily permeates the skin, penetrates the skin to directly increase skin elasticity of muscular nerves and thereby to show effects within a short time, prevents aging by regulating the secretion of catecholamines (adrenalin and noradrenalin), which are one of the main causes for wrinkling, and contributes to skin elasticity and beauty by temporarily controlling the signaling action of the SNARE complex (allows muscles to repeatedly contract and relax, and to secrete neurotransmitters, which acts to form wrinkles) to regulate repetitive movements of the wrinkles.

In addition, copper tripeptide-1 is a human-derived natural peptide found in human blood, saliva and the like.

Copper tripeptide-1 is bonded to albumin in the blood. Copper tripeptide-1 is present at about 300 ng/ml at age 20, but gradually decreases with aging and then reaches about 80 ng/ml at age 60. Such kappatripeptide-1 is a peptide created by proteolytic enzymatic degradation of cellular matrix proteins or inflammation-related proteins in the body when a skin injury occurs, which is known to remove damaged collagen or elastin generated by skin damage, inhibit scars, and promote skin regeneration by producing stem cells. Kappatripeptide-1 is also known to promote hair growth and reduce hair loss.

In addition, Avena sativa (Oat) kernel extract is extracted from the kernel of Avena sativa. The oat kernel contains ingredients such as niacin, sodium, proteins, carbohydrates, retinol, β-carotene, vitamin A, vitamin B1, vitamin B6, vitamin C, vitamin E, dietary fibers, zinc, folic acid, phosphorus, lipids, iron and potassium. Oat bark contains ingredients such as beta-glucan, saponin and dietary fiber. In particular, dietary fibers are present in 2 to 8-time greater amounts than red bean bark, rye bark, barley bark, millet bark and brown rice bark.

The reason why such an oat kernel extract is needed will be described below. Silicon dioxide, which is contained in a large amount in illite and sodium silicate, has an advantage of effectively removing waste and excess sebum in the pores with strong adsorption power, but may cause skin dryness because it may remove even oils and moisture in the skin due to strong adsorption power.

Therefore, in order to overcome such drawbacks, oats are used in the present invention. Owing to superior moisturizing effect of oats, it is possible to effectively remove waste and excessive sebum in the pores and reduce pores, without causing drying.

The second formulation is a supplemented nutrient, which, in particular, improves whitening effect of the skin.

Such a second formulation includes, based on 53.8 parts by weight of the illite extract, 1 part by weight of a Portulaca oleracea extract, 1 part by weight of a green tea extract, 2 parts by weight of a white flower complex, 2 parts by weight of sodium hydroxide (NaOH), 0.2 parts by weight of poly-epsilon-lysine and 40 parts by weight of sodium silicate.

The Portulaca oleracea extract is obtained by extracting Portulaca oleracea L., and Portulacea herba which is a hay of Portulaca oleracea L., which cures wounds, soothes the skin and is thus widely used to treat skin diseases and is generally used for cosmetics, ointments and wet tissues due to excellent antimicrobial and anti-inflammatory activities, and moisturizing power.

The green tea extract is extracted from the leaves of green tea. The polyphenol component (catechin), which is contained in a large amount in green tea, has been utilized in applications such as whitening, soothing and anti-aging for a long time owing to excellent cosmetic effects. In addition, the green tea extract has excellent anti-oxidation, odor removal (deodorization) and pore astringency.

The green tea extract contains great amounts of vitamins beneficial to the skin, for example, vitamin C, which removes skin spots such as stains and freckles, vitamin E, which suppresses skin aging, and vitamins A and B2, which are effective for treating acne.

In addition, the catechin ingredient promotes fat burning to discharge cholesterol outside the body, which is effective in controlling body weight and improving skin beauty.

In addition, the green tea extract promotes healing of skin wounds and is effective for preventing adult diseases based on vitamins and minerals.

Sodium hydroxide (NaOH) functions to regulate pH. When the pH is excessively high, the skin is irritated, and when the pH is excessively low, silicate may be excessively precipitated from sodium silicate. For this reason, pH control is needed.

Control of pH may be first carried out by the illite extract described below. When the illite extracts having a controlled pH according to a variety of Preparation Examples are used, sodium hydroxide controls a pH concentration when finally applied to a cosmetic pack, based on the pH value corresponding to the applied illite extract, to provide a pH concentration suitable for the cosmetic pack (or selected by a user), preferably pH of 9 to 13.

Depending on design conditions, the pH concentration is selectively controlled by the illite extract used in the present invention. For this reason, addition of sodium hydroxide may be omitted.

The amount of added sodium hydroxide, 2 parts by weight, may be replaced with the white flower complex, so that the amount of white flower complex corresponds to 4 parts by weight.

Poly-epsilon-lysine serves as a preservative to prevent the first formulation and/or second formulation from being spoiled.

Sodium silicate is a concentrated aqueous solution of sodium silicate (Na₂SiO₃, liquid) obtained by melting silicon dioxide (SiO₂) in alkali (in general, Na₂O), which is the same as general sodium silicate in that it is dissolved in water and alkali, except that the amount of SiO₂ is increased. It has a normal molar ratio of SiO₂/Na₂O of 2 to 3.

Such sodium silicate contains ingredients such as silicon dioxide (SiO₂), sodium oxide (Na₂O) and iron oxide (Fe₂O₃). Among them, silicon dioxide is present in the highest amount and a small amount of other insoluble solid ingredients is present.

As described above, silicon dioxide functions to remove waste and excessive sebum in pores, sodium oxide regulates moisture and osmotic pressure, and iron oxide acts on collagen binding.

In addition, sodium silicate is highly viscous, transparent, absorbs carbon dioxide in the air to precipitate gel-shaped silicate and exhibits strong adhesive strength and adsorptive strength.

That is, sodium silicate enables stable mixing of the composition of the first formulation and/or the second formulation. In addition, sodium silicate, which contains a high amount of silicon dioxide and has strong adsorptive strength, can effectively remove waste and excessive sebum in the pores.

The white flower complex prevents oxidation of skin and thus effectively provides skin whitening. Such a white flower complex is an extract which contains, as main ingredients, flowers of Ipomoea purpurrea L., Paeonia lactiflora, Lilium longiflorum, Magnolina liliflora desrouss and Convallaria keiskei miquel.

In addition, the extract may be used alone or in combination of one or more thereof.

However, according to the present invention, as a preferred material for better understanding, a mixture of the Ipomoea purpurea L., Paeonia lactiflora, Lilium longiflorum, Magnolina liliflora desrouss and Convallaria keiskei miquel which are mixed in the equal ratio (1:1:1:1:1) is used.

Seeds of Ipomoea purpurea L. contain pharbitin, nilic acid and gallic acid.

Such Ipomoea purpurea L. can be used in the form of an extract of a mixture of its seeds and egg whites, which helps to eliminate freckles, spots and age spots.

Paeonia albiflora pallas var. trichocarpa bunge [Paeonia japonica (Makino) Miyabe & Takeda] is a perennial plant which is also called “Peony roots”, alleviates blood vessels to facilitate circulation and eliminates couvercle to remove abscess. Paeonia albiflora pallas var. trichocarpa bunge also relieves abdominal pain, removes extravasated blood and removes pus.

Paeonia albiflora pallas var. trichocarpa bunge contains volatile oils, benzoic acid, Paeoniflorin, paeonol, peonin, fatty oils, resins, tannins, sugars, starch, mucilage, beta-sitosterol and triterpenoids. Accordingly, this helps to supply protein nutrients, inhibits skin spots, freckles and pigmentation, and contributes to whitening.

In addition, paeoniflorin facilitates blood circulation of the peripheral blood vessels and blood vessels distributed in the digestive tract mucosa, has strong antimicrobial activity, and has anti-inflammatory and antipyretic activities.

Lilium iongiflorum (Lilium candidum) is used in the form of the lily roots collected in the winter when the nutrients are gathered in the bulbs after the flowering season are used, and these lily roots are one of herbal medicines that have been used frequently since ancient times.

Lilium iongiflorum mainly contains carbohydrates, vegetable fats, minerals and vegetable proteins, so that it stabilizes the mind, cures insomnia, and is effective in improving retention of fluids, and relieving mental and physical stability after hematemesis or fever with a dry cough.

In addition, it is known to have an effect of assisting hemostasis of bloody phlegm or phlegm, and to be effective in soothing the skin and in imparting moisture to the dry skin by cooling and imparting humidity to the skin.

Magnolina liliflora desrouss is called “jade tree” because it is a clean and precious tree like jade, or is called “jade orchid” because a flower like jade emits an orchid-like scent, or is called “orchid tree” because it is an orchid-like tree, or is called “lotus tree” because it is a large and charming lotus blossomed on the tree.

Magnolina liliflora desrouss is effective for protecting the surface of the mucous membrane based on the convergence action, expanding capillary blood vessels, relieving inflammation, smoothing the breathing and alleviating symptoms, and its essential oil component has analgesic, soothing, blood pressure-controlling, and skin fungus and micrococcus-inhibiting activities.

Very weak skin inflammation may cause irritation, itching and erythema, while serious skin inflammation may cause skin damage. This is the result of biological reaction and is because oxidized substances are created and accumulated and then cause damage to most human body organs including cells. Accordingly, it is important to supply proper antioxidants in order to prevent aging. When such Magnolina liliflora desrouss is applied to the skin, it has similar antioxidant activity to 1.8% of vitamin C and thus has an effect of promoting blood circulation and antibacterial activity.

Convallaria keiskei Miquel (Convallaria majalis L.) has the scent of a flower and is thus called “scent orchid”, “Convallaria”, or “Grass of Convallaria”. It is called “lily of valley” in England, called “small bell” in Germany, and is sometimes called “stairway leading to heaven” from flowers blossoming one after another, in France.

Such Convallaria keiskei Miguel contains ingredients such as asparagine, caffeic acid, citric acid, convallarin, convallaric acid, convallamarin, majaloside, malic acid and rutin. For this reason, it is called Convallaria in oriental medicine and is used as a cardiovascular drug, a diuretic, a blood circulation promoter or the like, and is also used as a raw material of fragrance because of its fragrance.

A mixture of the first formulation and the second formulation in a ratio of 1:1 may be used as a pack composition. The pack composition may be provided in the form of a mixture of the first formulation and the second formulation (in the case of a mask pack or cream pack). Alternatively, when the pack composition is provided in the form of a cream pack, the first formulation and the second formulation are separately accommodated and the mix ratio of 1:1 may be stated.

### REFERENCE EXAMPLE 1

### Method for Preparing Illite Extract with Controlled pH

### 1) Preparation of Illite Powder

Illite was ground to prepare an illite powder.

An illite rock or boulder may be ground, grinding may be carried out by installing a rebound plate on the inner wall of a crusher and repeating hitting and bouncing, and a screen or grid may be further installed under the crusher to conduct screening.

Alternatively, the illite may be ground using turbo mills, super-micron mills, pot mills, tube mills, conical mills, jet grinders, disc mills, pin mills, stamp mills, shredders, hammer mills, hammer crushers, cone crushers, jaw crushers, cutter mills, edge runners, roller mills, Raymond mills, turbo micron mills or jet mills.

The illite may be ground into a powder with a size of 100 to 500 mesh and then used for extraction.

### 2) Pressurization and Heating

The illite powder was charged in an airtight container, water was added thereto, and the resulting mixture was pressurized and heated to prepare an illite extract.

At this time, water may be distilled water or purified water. Tap water or underground water is not preferably because they contain not only pure water molecules, but also mineral ingredients and various ions, which may affect pH during preparation of the illite extract.

The distilled water or purified water may be added in an amount of 2 to 4 parts by weight, based on 1 part by weight of the illite powder.

A pressure of 1 to 200 atm may be applied to the inside of the airtight container and heating may be carried out at a temperature of 100 to 400° C.

The heating time may be varied depending on design conditions and is from 1 to 5 hours.

### 3) Water Vapor Elution

The water vapor produced in the airtight container during pressurization and heating was eluted.

At this time, pH of the illite extract is changed depending on the time for which the water vapor stays in the container and the rate at which the water vapor is eluted. Accordingly, the most important feature to obtain an illite extract with a controlled pH is to determine water vapor elution conditions.

The water vapor may be eluted through a tube connected to the airtight container and the tube may have a diameter of 5 to 50 mm.

The water vapor is eluted through the connected tube and an illite extract is produced by the subsequent condensation step.

In addition, heating may be conducted in order to maintain the inner temperature of the airtight container constant during elution of water vapor.

When the time for which the water vapor stays in the container is increased by lengthening the heating time or decreasing the diameter of the tube, water vapor is compressed so that the concentration of ions extracted from the illite is increased. Accordingly, the pH of prepared illite extract is decreased.

On the other hand, when the time for which the water vapor stays in the container is decreased by shortening the heating time or increasing the diameter of the tube, the concentration of ions extracted from the illite is decreased so that the pH of prepared illite extract is increased.

That is, the pH of the illite extract decreases as the time for which the water vapor stays in the container is increased, and increases as the time for which the water vapor stays in the container decreases.

The pressure of eluted water vapor as well as the time for which the water vapor stays in the container may affect pH of the illite extract. Accordingly, the tube where water vapor is eluted is provided with a pressure control valve so that the valve is opened or closed depending on pressure and therefore, pH of the illite can be controlled more precisely.

For example, assuming that the pressure of the pressurized and heated airtight container is 20 atm, the pressure when the tube is opened during water vapor elution starts from 20 atm and gradually decreases, as water vapor is eluted.

At this time, the rate of decreasing pressure may be changed depending on the diameter of the tube. That is, when the diameter of tube is great, the pressure is rapidly decreased from 20 atm and water vapor is eluted. On the other hand, when the diameter of the tube is small, the pressure is slowly decreased, the time for which water vapor stays in the container increases, and the concentration of ion ingredients increases and an illite extract with low pH can thus be obtained.

When a pressure control valve is further provided in the tube where water vapor is eluted, pH can be controlled more precisely by obtaining an illite extract depending on pressure range.

For example, assuming that the pressure of the pressurized and heated airtight container is 20 atm, there occurs a difference in pH between an illite extract obtained by condensing water vapor from 20 atm to 10 atm immediately after opening, and an illite extract obtained by condensing water vapor having a pressure which gradually decreases from 10 atm and reaches the atmospheric pressure.

The illite extract obtained at a higher pressure has a higher water vapor compression proportion and thus lower pH, while the illite extract obtained at a lower pressure has a higher pH range.

That is, the pH of the illite extract is controlled depending on the diameter of the tube connected to the airtight container and eluting water vapor, and pH can be controlled more precisely through the pressure control valve equipped in the tube.

### 4) Water Vapor Condensation

The water vapor eluted from the tube connected to the airtight container is condensed to obtain an illite extract.

The condensation of water vapor may be carried out by an ordinary method, preferably a water cooling method using cooling water.

Through the step, the illite extract can be prepared and the illite extract with pH controlled to a desired level can be prepared by controlling the elution rate of water vapor.

### PREPARATION EXAMPLE 1

### Illite Extract with Controlled pH

200 g of an illite powder ground to a size of 100 to 500 mesh and 1,000 ml of distilled water were charged in an airtight container, and the resulting mixture was heated at 20 atm and at a temperature of 150 to 200° C. for 4 hours. The water vapor was eluted through a tube with a diameter of 5 mm from the airtight container and condensed with cooling water to obtain an illite extract.

### PREPARATION EXAMPLE 2

### Illite Extract with Controlled pH

200 g of an illite powder ground to a size of 100 to 500 mesh and 1,000 ml of distilled water were charged in an airtight container, and the resulting mixture was heated at 20 atm and at a temperature of 150 to 200° C. for 4 hours. The water vapor was eluted through a tube with a diameter of 10 mm from the airtight container and condensed with cooling water to obtain an illite extract.

### PREPARATION EXAMPLE 3

### Illite Extract with Controlled pH

200 g of an illite powder ground to a size of 100 to 500 mesh and 1,000 ml of distilled water were charged in an airtight container, and the resulting mixture was heated at 20 atm and at a temperature of 150 to 200° C. for 4 hours. The water vapor was eluted through a tube with a diameter of 20 mm from the airtight container and condensed with cooling water to obtain an illite extract.

### PREPARATION EXAMPLE 4

### Illite Extract with Controlled pH

200 g of an illite powder ground to a size of 100 to 500 mesh and 1,000 ml of distilled water were charged in an airtight container, and the resulting mixture was heated at 20 atm and at a temperature of 150 to 200° C. for 4 hours. The water vapor was eluted through a tube with a diameter of 35 mm from the airtight container and condensed with cooling water to obtain an illite extract.

### PREPARATION EXAMPLE 5

### Illite Extract with Controlled pH

200 g of an illite powder ground to a size of 100 to 500 mesh and 1,000 ml of distilled water were charged in an airtight container, and the resulting mixture was heated at 20 atm and at a temperature of 150 to 200° C. for 4 hours. The water vapor was eluted through a tube with a diameter of 50 mm from the airtight container and condensed with cooling water to obtain an illite extract.

### PREPARATION EXAMPLE 6

### Illite Extract with Controlled pH

200 g of an illite powder ground to a size of 100 to 500 mesh and 1,000 ml of distilled water were charged in an airtight container, and the resulting mixture was heated at 20 atm and at a temperature of 150 to 200° C. for 4 hours. The water vapor was eluted through a tube with a diameter of 10 mm from the airtight container and a pressure-control valve was further provided in the tube, to elute water vapor from 20 to 15 atm, and condense the same with cooling water, thereby to obtain an illite extract.

### PREPARATION EXAMPLE 7

### Illite Extract with Controlled pH

200 g of an illite powder ground to a size of 100 to 500 mesh and 1,000 ml of distilled water were charged in an airtight container, and the resulting mixture was heated at 20 atm and at a temperature of 150 to 200° C. for 4 hours. The water vapor was eluted through a tube with a diameter of 10 mm from the airtight container and a pressure-control valve was further provided in the tube, to elute water vapor from 15 to 10 atm, and condense the same with cooling water, thereby to obtain an illite extract.

### PREPARATION EXAMPLE 8

### Illite Extract with Controlled pH

200 g of an illite powder ground to a size of 100 to 500 mesh and 1,000 ml of distilled water were charged in an airtight container, and the resulting mixture was heated at 20 atm and at a temperature of 150 to 200° C. for 4 hours. The water vapor was eluted through a tube with a diameter of 10 mm from the airtight container and a pressure-control valve was further provided in the tube, to elute water vapor from 10 to 5 atm, and condense the same with cooling water, thereby to obtain an illite extract.

### PREPARATION EXAMPLE 9

### Illite Extract with Controlled pH

200 g of an illite powder ground to a size of 100 to 500 mesh and 1,000 ml of distilled water were charged in an airtight container, and the resulting mixture was heated at 20 atm and at a temperature of 150 to 200° C. for 4 hours. The water vapor was eluted through a tube with a diameter of 10 mm from the airtight container and a pressure-control valve was further provided in the tube, to elute water vapor from 5 to 1 atm, and condense the same with cooling water, thereby to obtain an illite extract.

### TEST EXAMPLE 1

### Measurement of pH of Illite Extract Depending on Diameter of Tube

In an attempt to identify the effect of controlling pH of the illite extract depending on the conditions of water vapor elution, pH of illite extracts prepared while changing the diameter of the tube associated with the rate of water vapor elution was measured.

pH of illite extracts prepared in Preparation Examples 1 to 5 was measured and measurement was conducted at the same temperature.

Results of pH measurement are shown in the following table 1.

TABLE 1

| | Prepa- | Prepa- | Prepa- | Prepa- | Prepa- |
|---|---|---|---|---|---|
| | ration | ration | ration | ration | ration |
| | Example 1 | Example 2 | Example 3 | Example 4 | Example 5 |
| pH | 4.75 | 5.52 | 6.41 | 7.68 | 9.07 |

Measurement results showed that the illite extract according to Preparation Example 1 had an acidic pH of 4.75, the illite extracts according to Preparation Examples 2 and 3 had weak acidic pH of 5.52 and 6.41, respectively, the illite extract according to Preparation Example 4 had a neutral pH of 7.68, and the illite extract according to Preparation Example 5 had a basic pH of 9.07.

It could be seen that pH of the illite extract was changed depending on the elution rate of water vapor and an illite extract that could control pH within a desired range was prepared.

### TEST EXAMPLE 2

### Measurement of pH of Illite Extract Depending on Pressure Range

In an attempt to identify the effect of controlling pH of the illite extract depending on the conditions of water vapor elution, pH of illite extracts prepared while changing the pressure of water vapor to be released was measured.

pH of illite extracts prepared in Preparation Examples 6 to 9 was measured and measurement was conducted at the same temperature.

Results of pH measurement are shown in the following table 2.

TABLE 2

| | Preparation | Preparation | Preparation | Preparation |
|---|---|---|---|---|
| | Example 6 | Example 7 | Example 8 | Example 9 |
| pH | 4.96 | 5.21 | 5.52 | 5.60 |

Measurement results showed that the illite extract according to Preparation Example 6 had a pH of 4.96, the illite extract according to Preparation Example 7 had a pH of 5.21, the illite extract according to Preparation Example 8 had a pH of 5.52 and the illite extract according to Preparation Example 9 had a pH of 5.21, which means that, as the pressure of eluted water vapor decreases, pH of the obtained illite extract increases.

Accordingly, by setting the elution pressure of water vapor, pH of the illite extract can be controlled more precisely.
When the composition was prepared using a conventional illite extract, pH was inevitably changed by other mixed materials.

However, since the method for extracting illite according to the present invention enables the illite to be extracted within the target pH range, the final composition having a suitable pH could be prepared by previously checking pH of the mixed material and thus controlling the pH of the illite extract to be mixed.

Accordingly, pH could be suitably controlled with only the illite extract without an additional pH controller.

For example, the pH of a composition used for the skin was known to be preferably a weak acidity of pH 5.5 which is similar to the skin. When the illite extract was used for a cosmetic composition, the illite extract could be extracted such that the cosmetic composition had a pH of 5.5.

In addition, the preferred pH of compositions used for skin may be changed depending on skin type. For example, pH of the composition suitable for atopic skin is about 6, and pH of the composition suitable for trouble skin is about 4.5 to 5.5. In this case, pH can be controlled well without an additional pH controller by using the illite extract having a controlled pH suitable for conditions.

### TEST EXAMPLE 3

### Measurement Test of White Flower Complex Efficacy: Tyrosinase Inhibition

### 3-1. Test Sample

The test sample used herein was 2 kg of a mixture of respective aforementioned extracts mixed in the same ratio.

The control group used herein was equal weights of Mulberry extract and albutin generally used as a whitening composition in cosmetics.

### 3-2. Test Method

The test method was an ordinary method to analyze inhibition of tyrosinase.

### 3-3. Test Results

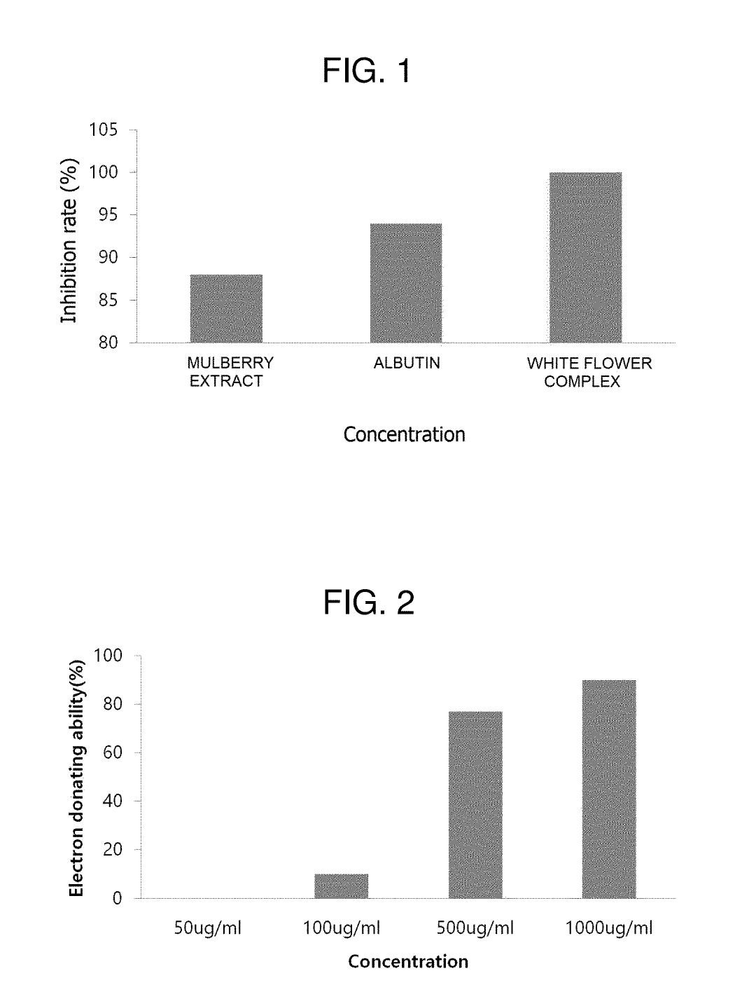

The test results in Test Example 3 were referenced from FIG. 1 of the attached drawings.

FIG. 1 shows results of measurement tests associated with tyrosinase inhibition effect of a white flower complex in a pack composition containing an illite extract as an active ingredient according to the present invention.

Referring to FIG. 1 of the attached drawings, as compared to control groups known to have excellent skin whitening effect, that is, Mulberry extract and albutin, the white flower complex could exhibit excellent tyrosinase inhibition.

These results showed that the white flower complex inhibited oxidation based on better tyrosinase inhibition, as compared to control groups. For this reason, the white flower complex was considered to exhibit excellent skin whitening effects, as compared to control groups.

### TEST EXAMPLE 4

### Measurement Test of White Flower Complex: Antioxidant Activity

### 4-1. Test Sample

Test samples were white flower complexes added in amounts of 50 μg/ml, 100 μg/ml, 500 μg/ml and 1000 μg/ml, with respect to 1 ml of the illite extract contained in the second formulation.

### 4-2. Test Method

The test method used herein was DPPH scavenging activity analysis generally used in the art, and IC50 of the test sample was measured.

### 4-3. Test Results

The test results in Test Example 4 were referenced from FIG. 1 of the attached drawings.

FIG. 2 shows test results demonstrating that, regarding DPPH free radical scavenging activity of the white flower complex in the pack composition containing an illite extract as an active ingredient according to the present invention, the feature that the effective ingredients of the white flower complex have an effectiveness probability of 50% is associated with the content of the white flower complex.

The effective ingredients mean ingredients contained in respective flowers described as the white flower complex.

FIG. 2 of the attached drawing showed that, when the white flower complex is contained in an amount of 100 μg/ml, the effective ingredients of the white flower complex have an effectiveness probability of 50%, in particular, from the content of 500 μg/ml, the probability significantly increases.

This indicates that, as the content of the white flower complex increases, the probability that its effective ingredients are effective at 50% gradually increases. For this reason, as described above, DPPH free radical scavenging activity as well as the antioxidant activity of the white flower complex increases.

These test results indicate that, as the content of the white flower complex increases, the aforementioned effects are improved. For this reason, when the white flower complex according to the present invention is present in an amount of 2 parts by weight, antioxidant activity can be further improved.

In addition, based on antioxidant activity-associated results, melanin synthesis is inhibited by antioxidant activity of the skin and skin brightening effect can be improved by reduction of melanin, so that skin whitening effect can be improved by the white flower complex according to the present invention.

Whether or not this skin whitening relates to the content of the white flower complex was identified by sensory evaluation.

### TEST EXAMPLE 5

### Sensory Evaluation

### 5-1. Test Method

In Experimental Example 5, adult Mongoloid women of 20 to 30 years old with healthy skin conditions participated in the test as panelists for 30 days.

In addition, the panelists were asked to avoid exposure of the skin to UV as much as possible and to apply UV-blocking products to the skin or cover the corresponding site with clothing, when going out.

The present test targeted skin sites of the human body that can be covered by clothing and irritation by UV could be minimized because the present test was conducted at the change of seasons and the skin sites were thus covered with clothing.

In addition, Test groups include Example 1 as well as Comparative Examples 1, 2 and 3 as control groups, and results can be seen from the following Table 3.

### 5-2. Test Results

Test results of Test Example 5 are shown in the following table 3.

TABLE 3

| | | Compar- | Compar- | | Compar- |
|---|---|---|---|---|---|
| | | ative | ative | | ative |
| | | Exam- | Exam- | Exam- | Exam- |
| Item | Composition | ple 1 | ple 2 | ple 1 | ple 3 |
| First | Illite extract | 85.76 | 85.76 | 85.76 | 85.76 |
| formu- | Carbomer | 2.0 | 2.0 | 2.0 | 2.0 |
| lation | Adenosine | 0.04 | 0.04 | 0.04 | 0.04 |
| | Caffeine | 1.2 | 1.2 | 1.2 | 1.2 |
| | β-glucan | 1.2 | 1.2 | 1.2 | 1.2 |
| | Hovenia dulcis | 0.6 | 0.6 | 0.6 | 0.6 |
| | fruit extract | | | | |
| | Green tea extract | 0.6 | 0.6 | 0.6 | 0.6 |
| | Whitening | 0.6 | 0.6 | 0.6 | 0.6 |
| | composition | | | | |
| | Black soybean | 0.5 | 0.5 | 0.5 | 0.5 |
| | extract | | | | |
| | Oreganum | 0.5 | 0.5 | 0.5 | 0.5 |
| | vulgare extract | | | | |
| | Scutellaria | 0.2 | 0.2 | 0.2 | 0.2 |
| | baicalensis | | | | |
| | extract | | | | |
| | Sophora | 0.2 | 0.2 | 0.2 | 0.2 |
| | angustifolia | | | | |
| | extract | | | | |
| | Acetyl | 3.0 | 3.0 | 3.0 | 3.0 |
| | hexapeptide | | | | |
| | Copper | 2.0 | 2.0 | 2.0 | 2.0 |
| | tripeptide | | | | |
| | Avena sativa | 1.0 | 1.0 | 1.0 | 1.0 |
| | (Oat) kernel | | | | |
| | extract | | | | |
| | Octanediol | 0.3 | 0.3 | 0.3 | 0.3 |
| | Phenoxyethanol | 0.3 | 0.3 | 0.3 | 0.3 |
| Second | Illite extract | 54.8* | 54.3* | 53.8** | 53.3* |
| formu- | Portulaca | 1.0 | 1.0 | 1.0 | 1.0 |
| lation | oleracea extract | | | | |
| | Green tea extract | 1.0 | 1.0 | 1.0 | 1.0 |
| | White flower | 1.0* | 1.5* | 2.0** | 2.5* |
| | complex | | | | |
| | Sodium | 2.0 | 2.0 | 2.0 | 2.0 |
| | hydroxide | | | | |
| | Poly-epsilon- | 0.2 | 0.2 | 0.2 | 0.2 |
| | lysine | | | | |
| | Sodium silicate | 40.0 | 40.0 | 40.0 | 40.0 |
| Evalua- | Satisfaction of | unsatisfied | normal | satisfied | normal |
| tion | panelists | | | | |
| | Visible difference | unchanged | Slightly | changed | changed |
| | | | changed | | |

As can be seen from Table 3 above, in Comparative Example 1, most panelists were unsatisfied with the texture of the pack used and did not have skin whitening effect. There were no panelists who showed visible changes.

The term “visible changes (differences)” used herein was evaluated by obtaining an image of the skin before testing, obtaining an image of the skin after testing and comparing the skin tone (chromaticity) between before and after testing.

In Comparative Example 2, the panelists were generally satisfied with the texture of pack used and there was a conflict of opinions between some panelists who had skin whitening effect and other panelists who did not had skin whitening effect. Also, like the opinion about skin whitening, there were both panelists who underwent visible changes and panelists who did not undergo visible changes.

In Example 1, almost all panelists were satisfied with texture and skin whitening effect. Also, regarding skin tone of all panelists, the improved skin tone of about 90% of the panelists was recognized at first and about 10% of the panelists were shown to have skin tone improvement by comparing the skin tone between the images before and after testing.

In Comparative Example 3, most panelists had excellent skin whitening effect and showed visibly remarkable difference.

However, unlike the whitening effect, about 40% of the panelists had heavier texture to the skin than conventional other products and discomfort due to the feeling of leaving more residues on the skin after use. 20% of the panelists also had the same opinion after discussion.

This was considered because moisture content is decreased as the content of the white flower complex is increased and the content of illite extract is decreased.

Accordingly, it could be seen that, when the white flower complex is increased to 2 parts by weight or more, in order to increase effect of the white flower complex, there occurred discomforts upon use, and when the white flower complex was decreased to 2 parts by weight or less, the whitening effect occurring at the same time was less, or the time at which the whitening effect occurred become slow.

1. A pack composition containing an illite extract as an active ingredient comprising:
a first formulation having skin enhancement effect; and
a second formulation having skin whitening effect,
wherein the first formulation and the second formulation are mixed in the same ratio,
wherein the first formulation comprises:
based on 85.76 parts by weight of the illite extract,
2 parts by weight of carbomer;
0.04 parts by weight of adenosine;
1.2 parts by weight of caffeine;
1.2 parts by weight of β-glucan;
0.6 parts by weight of a Hovenia dulcis fruit extract;
0.6 parts by weight of a green tea extract;
0.6 parts by weight of a whitening composition;
0.5 parts by weight of a black soybean extract;
0.5 parts by weight of an Oreganum vulgare extract;
0.2 parts by weight of a Scutellaria baicalensis extract;
0.2 parts by weight of a Sophora angustifolia extract;
3.0 parts by weight of acetyl hexapeptide-8;
2.0 parts by weight of copper tripeptide-1;
1.0 part by weight of an Avena sativa (Oat) kernel extract;
0.3 parts by weight of octanediol (dermosoft® Octiol); and
0.3 parts by weight of phenoxyethanol, and
the second formulation comprises:
based on 53.8 parts by weight of the illite extract,
1 part by weight of a Portulaca oleracea extract;
1 part by weight of a green tea extract;
2 parts by weight of a white flower complex;
2 parts by weight of sodium hydroxide (NaOH);
0.2 parts by weight of poly-epsilon-lysine; and
40 parts by weight of sodium silicate.

2. The pack composition according to claim 1, wherein the whitening composition comprises one or more of a Centella asiatica extract, a Diospyros kaki leaf extract, a Theobroma cacao extract, a chamomile extract and a wine extract.

3. The pack composition according to claim 1, wherein the white flower complex comprises an extract of one or more selected from Ipomoea purpurea L., Paeonia lactiflora, Lilium longiflorum, Magnolina liliflora desrouss and Convallaria keiskei Miquel.

4. The pack composition according to claim 1, wherein pH of the illite extract can be controlled by preparing the illite extract using a method comprising the following steps:
grinding an illite boulder to a size of 100 to 500 mesh to prepare an illite powder;
adding the illite powder and water to an airtight container, and pressurizing and heating the resulting mixture at a pressure of 1 to 200 atm and at 100 to 400° C.;
eluting water vapor through a tube being connected to the airtight container and having a diameter of 5 to 50 mm, wherein the elution is carried out by controlling the diameter of the tube and controlling a pressure of the water vapor such that the water vapor having a pressure range set by a pressure-control valve is eluted; and
condensing the eluted water vapor to obtain the illite extract.

5. The pack composition according to claim 1, wherein the white flower complex is added in an amount of 500 to 1000 μg/ml, based on 1 mL of the illite extract, and
the white flower complex has tyrosinase inhibition and antioxidant effects.

6. A pack comprising the composition according to claim 1.
